(19) United States
(12) Patent Application Publication
(10) Pub. No.: US 2022/0378967 A1
(43) Pub. Date: Dec. 1, 2022

(54) RADIO FREQUENCY IDENTIFICATION PROCESS INDICATOR AND READER

(71) Applicant: STERIS Corporation, Mentor, OH (US)

(72) Inventors: Anthony James Fiorello, Mentor, OH (US); Michael Anthony Bacik, Fairview, OH (US); William Andrew Yirava, Willoughby, OH (US)

(21) Appl. No.: 17/826,549

(22) Filed: May 27, 2022

Related U.S. Application Data
(60) Provisional application No. 63/193,853, filed on May 27, 2021.

(51) Int. Cl. A61L 2/28; G01R 29/08; A61L 2/24; H01Q 1/42; H01Q 7/00; H01Q 1/38

(52) U.S. Cl. CPC A61L 2/28; G01R 29/0871; A61L 2/24; H01Q 1/422; H01Q 7/00; H01Q 1/38; A61L 2202/14; A61B 90/98

(57) ABSTRACT

Disclosed embodiments can pertain to process indicator enhancements. A process indicator can include a dye material that has electrical properties such as conductivity and capacitance that can be employed to determine whether observed conditions are acceptable or not to sterilize medical equipment. Furthermore, the indicators can employ amplification, a specialized antenna, or both to allow signals, such as an electronic property value, to be communicated through sterilization containers that can shield or degrade signals. A number and location of indicators can also be determined and utilized to assist in visual inspection of the indicators as well as automatic evaluation.

### CROSS-REFERENCE TO RELATED APPLICATIONS

This application claims the benefit of U.S. Provisional Application No. 63/193,853, filed May 27, 2021, and entitled “Radio Frequency Identification Process Indicator and Reader,” the entirety of which is incorporated herein by reference.

### BACKGROUND

Medical equipment can be reused after sterilization. Sterilization subjects the equipment to a lethal environment, such as heat and steam, to remove, kill, or deactivate bacteria and other microorganisms. Surgical equipment, such as forceps and scalpels, can be enclosed within a container or tray using a lid or sterilization wrap. The container can then be positioned in an autoclave, which is a machine that sterilizes the equipment by creating and subjecting the equipment to a lethal process. The equipment is then made available for reuse. The sterilization wrap and container lids can protect the sterile equipment from airborne contaminants prior to use.

### SUMMARY

The following presents a simplified summary to provide a basic understanding of some aspects of the disclosed subject matter. This summary is not an extensive overview. It is not intended to identify key/critical elements or to delineate the scope of the claimed subject matter. Its sole purpose is to present some concepts in a simplified form as a prelude to the more detailed description presented later.

Briefly described, aspects of the subject disclosure pertain to process indicators that can record conditions for use in determining whether medical equipment has been sterilized. The process indicators can employ a dyed material that provides a visual indication of conditions with respect to what is acceptable and unacceptable. In accordance with one aspect, the dyed material can include electrical properties such as resistance and capacitance that can be evaluated to permit automatic and non-visual evaluation of the process indicator. Further, the process indicator can include support for radio frequency communication, such as radio frequency identification (RFID) technology. The electrical properties can be determined and communicated to a reader that can evaluate the properties with respect to one or more thresholds to determine whether the conditions observed were acceptable or not for a sterilization process. Subsequently, the reader can communicate acceptance/rejection, pass/fail, or the like through a display. In one instance, an amplifier or specialized antenna can be employed and optionally included with an indicator to enhance a communication signal that may be shielded or degraded by a sterilization container or contents thereof. Furthermore, a reader can determine the number of indicators in a container and their location for use in an automatic process as well as human visual inspection of process indicators.

According to one aspect, disclosed embodiments can include a process indicator system that comprises a dye material that has a melting point at a predetermined sterilization temperature and can be moved along a defined path by steam that alters an electronic property value of a process indicator, a radio frequency antenna that communicates the electronic property value of the process indicator wirelessly, and an amplifier coupled with the radio frequency antenna that amplifies the radio frequency. The system can further comprise a flexible metal wire of a predetermined length that couples the radio frequency antenna to the amplifier at a distance. In one instance, the dye material conducts electricity. Further, the electronic property can be resistance, and resistance between two metal portions can vary based on a position of the dye material along the defined path. In this instance, the system can also comprise a first dielectric polymer layer, wherein the two metal portions and the dye material are overlaid on the first dielectric polymer layer, and a second dielectric polymer layer overlaid on the two metal portions and the dye material. Alternatively, the dye material can have a capacitive property. Further, the system can comprise a first metal layer, a first dielectric polymer layer overlaid on the first metal layer, the dye material overlaid on the first dielectric polymer layer, a second dielectric polymer layer overlaid on the dye material, and a second metal layer overlaid on the second dielectric polymer layer. The electronic property can be capacitance and the capacitance between the first metal layer and the second metal layer can vary based on a position of the dye material along the defined path. The system can also comprise a printed indication of a location of the dye material on the path that indicates that a sterilization condition has been met. Further, the system can comprise a power source that powers broadcast of the electronic property value.

In accordance with another aspect, disclosed embodiments can include a method comprising executing, on a processor, instructions that cause the processor to perform operations associated with process indicator evaluation. The operations comprise reading an amplified radio frequency of an electronic property value of a process indicator from an antenna coupled to the process indicator inside a sterilization container, wherein the process indicator includes an ink material with a melting point at a predetermined sterilization temperature that moves along a predefined path and alters the electronic property value, evaluating the electronic property value with respect to one or more predetermined thresholds, and conveying, for display on a display device, either pass or fail based on a result of the evaluating. In one instance, evaluating the electronic property value comprises comparing the electronic property value of resistance to a predetermined resistance threshold that indicates conditions for sterilization were satisfied. The operations can further comprise reading the electronic property value from a radio frequency antenna outside the sterilization container connected by a flexible wire to the process indicator inside the sterilization container. The operations can further comprise determining a number of process indicators inside the sterilization container through wireless communication, evaluating the electronic property value of the number of process indicators inside the sterilization container, and conveying fail as the result when any one of the number of process indicators fails. Further, the operations comprise determining a location of the process indicator within the sterilization container based on reading position and signal strength and conveying, for display on the display device, the location of the process indicator.

According to yet another aspect, disclosed embodiments can include a process indicator apparatus. The process indicator apparatus can include a dye material that has a melting point at a predetermined sterilization temperature and can be forced along a defined path by steam that alters an electronic property value of a process indicator, a radio frequency antenna that communicates the electronic property value of the process indicator wirelessly, and an amplifier that amplifies the radio frequency, in which the amplifier is coupled to the antenna with a predetermined length of flexible wire that allows the antenna to be positioned outside a sterilization container while the dye material and amplifier are inside the sterilization container. In one instance, the electronic property is resistance, and the resistance between two metal portions varies based on a position of the dye material along the defined path. In another instance, the electronic property is capacitance and the capacitance between a first metal layer and a second metal layer varies based on the position of the dye material along the defined path.

To the accomplishment of the foregoing and related ends, certain illustrative aspects of the claimed subject matter are described herein in connection with the following description and the annexed drawings. These aspects are indicative of various ways in which the subject matter may be practiced, all of which are intended to be within the scope of the disclosed subject matter. Other advantages and novel features may become apparent from the following detailed description when considered in conjunction with the drawings.

### BRIEF DESCRIPTION OF THE DRAWINGS

FIG. 1 illustrates an overview of an example implementation.

FIG. 2 is a block diagram of a representative process indicator.

FIGS. 3A-C are illustrations of varying states of a visual indicator.

FIGS. 4A-B illustrate resistive and capacitive indicator structures.

FIGS. 5A-B depict readers associated with resistive and capacitive indicators.

FIG. 6 is a block diagram of an extended antenna aspect.

FIG. 7 is a flow chart diagram of a method of verifying sterilization.

FIG. 8 is a flow chart diagram of a method of sterilization verification.

FIG. 9 is a schematic block diagram illustrating a suitable operating environment for aspects of the subject disclosure.

### DETAILED DESCRIPTION

Medical equipment can be sterilized before use. The medical equipment can be placed in a container or tray and inserted into a sterilization machine that creates a lethal environment that removes, kills, or deactivates bacteria or other microorganisms. One or more process indicators can be included within containers to record whether or not sterilization conditions have been met. In other words, the indicators can record data regarding the environment experienced by the medical equipment in the container, such as temperature and steam.

The container, wrapped or not, can be made available for use. A nurse, physician assistant, or other medical staff opens the container and visually inspects the indicators to verify that the medical equipment has been properly sterilized. Inspection of the indicators could be just prior to a procedure. If the indicators are converted, or in other words, have passed, the procedure can move forward. If not, another container of equipment needs to be acquired if available. Otherwise, the original equipment will need to be sterilized. Meanwhile, a patient could be anesthetized or otherwise prepared for surgery. As such, the patient may not be in a good position for an extended period of time should medical equipment be unavailable.

In one instance, a process indicator can be dyed material-based. The dyed material undergoes exposure to temperature and pressure that moves the dyed material along a chromatography paper to indicate pass or fail at the end of the cycle. For example, a layer of a multilayer indicator can comprise a dyed material pellet that resides in a cavity in a bottom layer. The dyed material pellet can have a melting temperature at or around a target sterilization temperature of a machine. The melted dyed material can then be moved by penetrating steam across the paper. A person can then visually inspect the indicator to determine whether pass or fail is specified. In some instances, the visual inspection may be difficult. For example, it may be difficult to see where the dyed material terminates. This difficulty can be exacerbated if the case is borderline pass/fail or the individual is visually impaired.

Details provided herein generally pertain to enhanced sterilization process indicators. In one instance, the indicators can include radio frequency communication. Stated differently, the indicators can incorporate or otherwise employ radio frequency identification (RFID) technology. RFID technology allows a determination to be made regarding whether or not a container of medical supplies experienced sterilization conditions without opening the container. This enables sterilization processors or technicians to verify that a container has passed by remotely interrogating the indicators before making the medical equipment available for use. In one instance, the indicators can also include a visual indicator to allow visual verification that an indicator has passed. However, a processing facility can have high confidence that medical equipment made available for use will not be returned with a tag indicative of failure. Further, an RFID reader can be employed after the container is open to aid in clarifying whether an indicator specifies pass or fail. A container and the medical instruments within a container can shield, degrade, or otherwise interfere with a radio communication of an indicator. To address this problem, amplification, a specialized antenna, or both can be employed for each individual indicator or a collection of indicators within a container. Multiple indicators can also be included in a container resulting in a final determination based on a result of all indicators in the container. The number of indicators in a container is often unknown to the end-user of the medical equipment. Through the use of RFID, the number and optionally location of each indicator in a container can be determined and presented. This is useful for a staff member opening the container to at least know the quantity and location of indicators for visual inspection.

Various aspects of the subject disclosure are now described in more detail with reference to the annexed drawings, wherein like numerals generally refer to like or corresponding elements throughout. It should be understood, however, that the drawings and detailed description relating thereto are not intended to limit the claimed subject matter to the particular form disclosed. Rather, the intention is to cover all modifications, equivalents, and alternatives falling within the spirit and scope of the claimed subject matter.

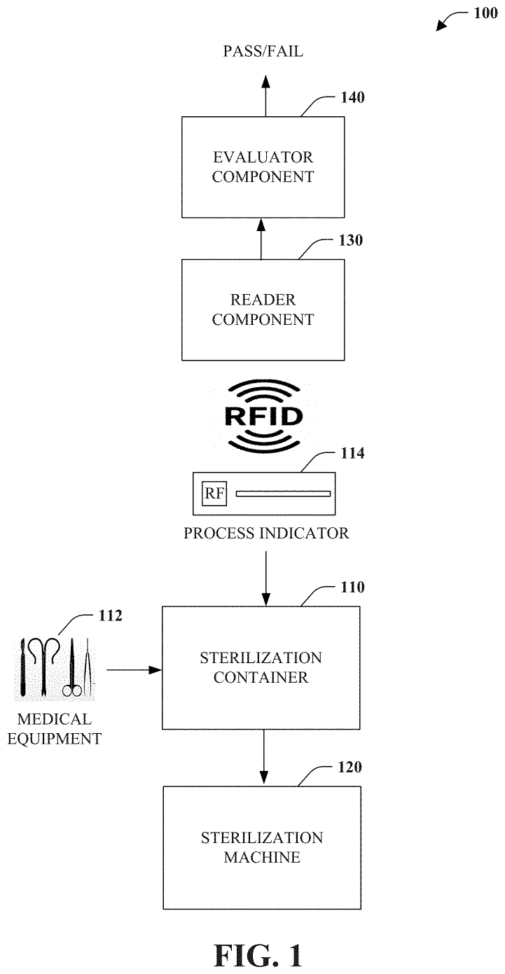

Referring initially to FIG. 1, a high-level overview of an example implementation is illustrated and described. As depicted, the implementation includes a sterilization container 110. The sterilization container 110 can be a box, tray, or other structure. The sterilization container 110 can include medical equipment 112, such as forceps and scalpels. Further, the sterilization container can include one or more process indicators 114. The sterilization container 110 is provided to sterilization machine 120, such as an autoclave. The sterilization machine 120 is operable to create an environment that sterilizes the medical equipment through heat and steam. The process indicators 114 within the sterilization container 110 can measure the characteristics experienced within the sterilization machine including temperature and steam exposure. For example, a process indicator 114 can include a dyed material pellet that melts at a predetermined sterilization temperature and moves across chromatography paper when penetrated by steam. In accordance with one aspect of the subject disclosure, the dyed material can have electrical properties such as resistance or capacitance that can be measured and utilized to determine whether the indicator tag specifies pass or fail. Furthermore, the process indicator 114 can include radio frequency technology such as radio frequency identification (RFID) that allows active or passive communication with a reader.

The reader component 130 is operable to acquire RFID-based communications from the process indicator 114. In one instance, when in range, the reader 130 can simply receive communications transmitted actively by the process indicator 114. Alternatively, the RFID reader 130 can interrogate a passive process indicator 114 to acquire data. Actively or passively communicated data can be provided to evaluator component 140. The evaluator component 140 is operable to determine whether the process indicator 114 specifies pass or fail with respect to sterilization. This determination can be done by comparing predetermined values associated with a pass or fail to actual data returned by the process indicator 114. For example, the dyed material can have a resistive characteristic that changes the resistance of an electrical circuit in a predictable manner when the indicator specifies pass as opposed to fail. Using this information, the evaluator 140 can be configured to decide based on comparing a circuit's resistance given particular thresholds. The evaluator 140 can operate similarly for capacitance, in which there is a predictable difference in capacitance for a process indicator that specifies pass versus fail.

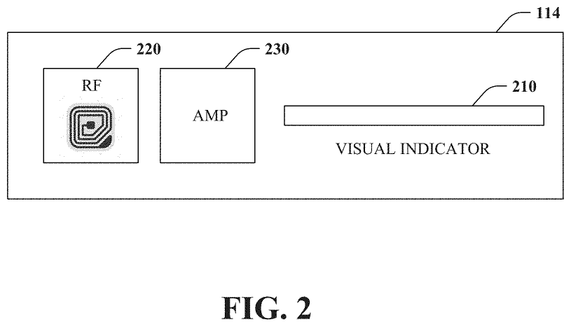

FIG. 2 illustrates a sample process indicator 114 in further detail. The process indicator 114 includes a visual indicator 210 that visually distinguishes between passing and failing with respect to exposure to conditions of a sterilization process. Stated differently, the visual indicator 210 allows an individual to see whether or not the process indicator is suggestive of sterilization or not. The process indicator 114 also includes a radio frequency component 220 that enables active or passive data communication over a radio frequency or the like. In this way, the process indicator 114 can remotely communicate a result or characteristics associated with a result. The process indicator 114 also includes an amplifier 230. The amplifier 230 is configured to amplify or strengthen the radio frequency associated with the radio frequency component 220. In one instance, the amplifier 230 can include a power source such as a standard or rechargeable battery. In another instance, the amplifier 230 can acquire and store power from a reader and utilize that power to amplify the radio frequency. For example, the amplifier can store power in a capacitor that can be used for amplification. In this manner, the radio frequency can be provided and read even when a container or equipment within the container interferes with the radio frequency.

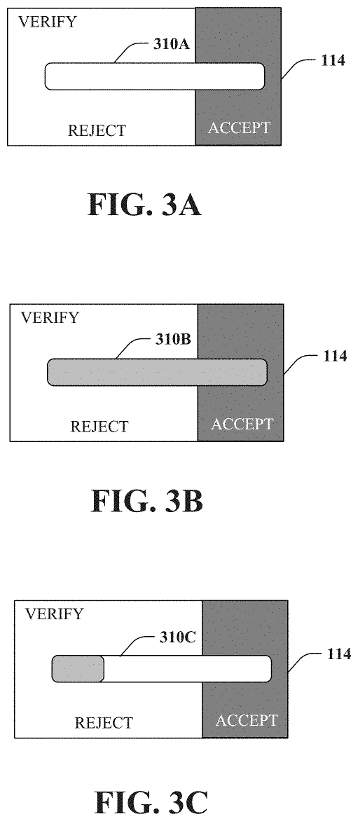

FIGS. 3A-C illustrates three process indicators 114 or tags with differing visual indicators 310A-C. Each process indicator 114 monitors a process to provide objective evidence that specific conditions have been met. In one embodiment, an ink or dye material is designed with a melting point around a particular sterilization temperature. When the melting point is reached for a given time, the solid ink melts and can be propelled or pushed by steam, for example, from one side to another. If the conditions of the process have been met, a visual color change can be viewed in a visible indicator. If conditions are not met or incomplete, no color change will be visible. Further, the process indicator 114 can be made of a material for color change with resistive or capacitive properties that can be exploited to read the process indicator electronically.

FIG. 3A depicts a process indicator 114 before a sterilization process. As shown, the visual indicator 310A is blank, white, or otherwise lacking in color. FIG. 3B and FIG. 3C indicate possible states after a process is complete. FIG. 3B illustrates a process indicator 114 that includes a visual indicator 310B that is completely colored in gray indicative that a process is acceptable. In other words, a sterilization process successfully achieved predetermined lethality conditions (e.g., temperature, duration). FIG. 3C depicts a process indicator 114 in which the visual indicator 310C that is only partially colored, capturing a process that should be rejected for failure to meet conditions. For example, the process may not have reached a particular temperature or maintained the temperature for a long enough. Alternatively, a non-condensable gas may have been present surrounding the indicator that prevented steam from fully reaching the process indicator.

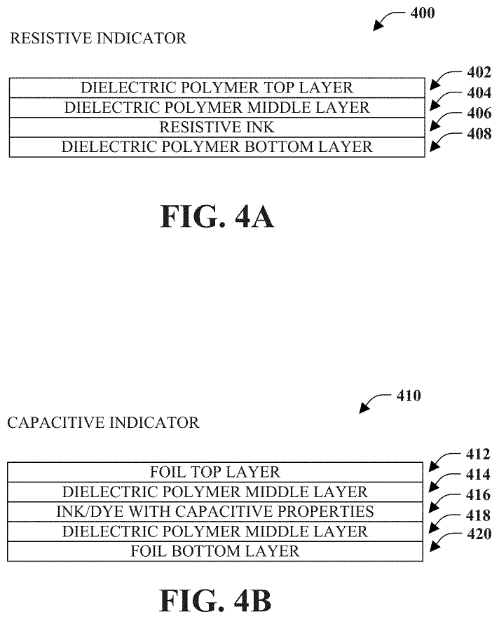

FIGS. 4A-B depict an example physical structure associated with a resistive and capacitive process indicator. The process indicator can be made of material for color change that has resistive or capacitive properties. The process indicator can be attached to a passive or active radio frequency identification tag to allow the resistance or capacitance to be read wirelessly.

In FIG. 4A, the indicator structure 400 is resistive. The indicator structure 400 can include a number of dielectric layers and resistive ink. As shown, the example indicator structure 400 includes a dielectric polymer top layer 402, dielectric polymer middle layer 404, resistive ink material 406, and dielectric polymer bottom layer 408. The resistive ink material 406 is positioned between dielectric layers that are electric insulators and inhibit current flow. The resistive ink material 406 is conductive and allows current to flow but reduces the current flow to a set amount based on a resistive property of the material. The resistive ink material 406 can be melted and pushed across an indicator. Before moving to an acceptable region, the resistance prohibits current flow. Once the resistive ink moves to an acceptable region, current can flow, and an output current can be equal to the input current reduced by a resistive factor of the ink material.

In FIG. 4B, the indicator structure 410 corresponds to a capacitive indicator. The indicator structure 410, as depicted, can include a foil top layer 412, dielectric polymer middle layer 414, ink with capacitive properties 416, a second dielectric polymer middle layer 418, and a foil bottom layer 420. There could also be a side-to-side foil arrangement. The foil top layer 412 and foil bottom layer 420 correspond to conductive plates of a capacitor that are separated by two dielectric layers 414 and 418, as well as an ink or dye with capacitive properties. The capacitance captured by the foil top layer 412 and foil bottom layer can indicate pass or failure. The ink or dye has capacitance properties that can affect the capacitance as it is moved or pushed across an indicator. For example, as the ink is moved across the indicator, the dielectric constant can be increased between the plates, producing a higher capacitance than otherwise. A capacitance higher than a predetermined threshold can indicate a passed state. A capacitance lower than a predetermined threshold can denote a failed state.

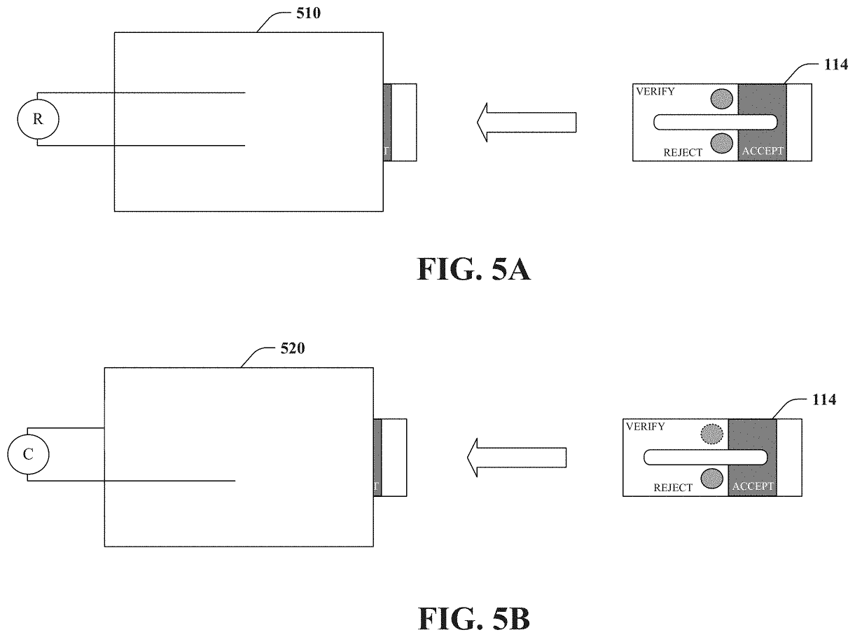

FIGS. 5A-B illustrate readers associated with resistive and capacitive indicators. In accordance with one aspect, the process indicators can be coupled with active or passive radio frequency identification technology to allow contactless communication. However, the process indicators 114 can be designed to enable contact-based communication.

FIG. 5A shows a resistive process indicator 114 and a resistance reader 510. Here, the process indicator 114 can correspond to the resistive indicator 400 of FIG. 4A. The process indicator 114 is placed inside or near the reader. The resistance is measured when the dyed material contacts the bare foil pads on adjacent points on the process indicator 114. In one example instance, the process indicator 114 is deemed to have passed if the resistance is lower than a predetermined threshold and failed if the resistance is higher than a predetermined threshold.

In FIG. 5B, the process indicator 114 can correspond to a capacitive indicator read by a capacitive reader 520. The process indicator 114 can correspond to the capacitive indicator 410 of FIG. 4B and can be placed inside the reader. The capacitance can be measured by contacting bare foil pads on opposite sides of the process indicator 114. The process indicator 114 can be deemed to have passed if the capacitance is higher than a predetermined threshold and failed if the capacitance is lower than a predetermined threshold.

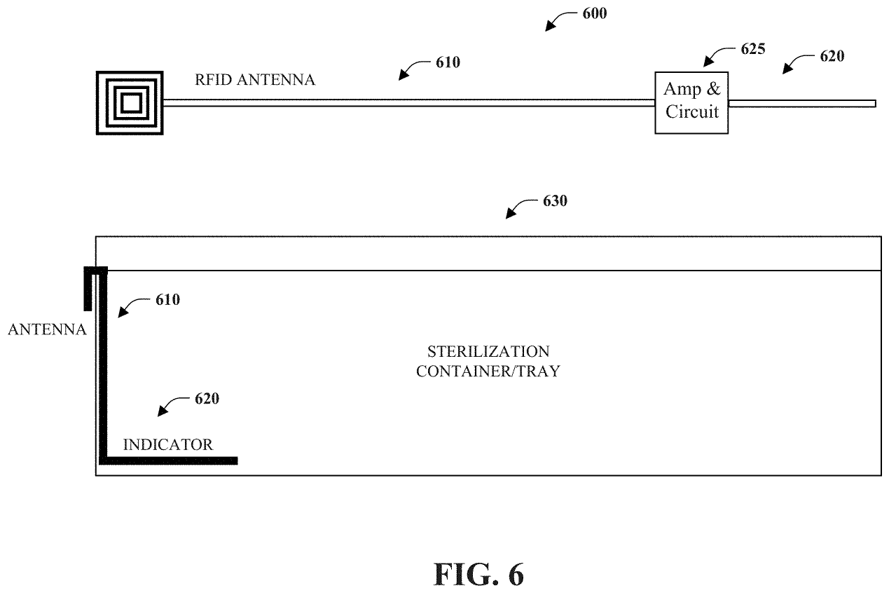

FIG. 6 is a block diagram of a system 600 including an extended antenna. Sterilization containers can be constructed of materials that interfere with or degrade radio frequency communication. In one instance, an amplifier can be employed on the radio frequency to aid in reading a process indicator wirelessly outside a sterilization container. Another embodiment is to employ an antenna that extends outside the container alone or in combination with an amplifier. As depicted, a process indicator 620 can be connected with an RFID antenna 610 that extends outside a sterilization container 630. The antenna can be made of thin, flexible material. Further, a signal to or from the process indicator 620 can be amplified by an amplifier and circuitry 625. The amplifier and circuitry can be embedded within the process indicator 620 or reside external but connected to the process indicator 620. In the example embodiment shown, the process indicator 620 or tag can be placed near the sides of the sterilization container or tray 630. In this manner, the antenna can be extended outside with good reception and eliminate signal reduction or degradation from metal or other materials of the sterilization container or tray 630.

The aforementioned systems, architectures, platforms, environments, or the like have been described with respect to interaction between several components. It should be appreciated that such systems and components can include those components or sub-components specified therein, some of the specified components or sub-components, and/or additional components. Sub-components could also be implemented as components communicatively coupled to other components rather than included within parent components. Further yet, one or more components or sub-components may be combined into a single component to provide aggregate functionality. Communication between systems, components, or sub-components can be accomplished in accordance with either a push or pull control model. The components may also interact with one or more other components not specifically described herein for brevity but known by those of skill in the art.

Various portions of the disclosed systems above and methods below can include or employ artificial intelligence, machine learning, or knowledge or rule-based components, sub-components, processes, means, methodologies, or mechanisms (e.g., support vector machines, neural networks, expert systems, Bayesian belief networks, fuzzy logic, data fusion engines, classifiers). Such components, among others, can automate certain mechanisms or processes performed thereby making portions of the systems and methods more adaptive as well as efficient and intelligent. For example, such mechanism can be employed by a process indicator reader to infer or predict the presence or absence of a process indicator, the location of a process indicator, and the value of a process indicator.

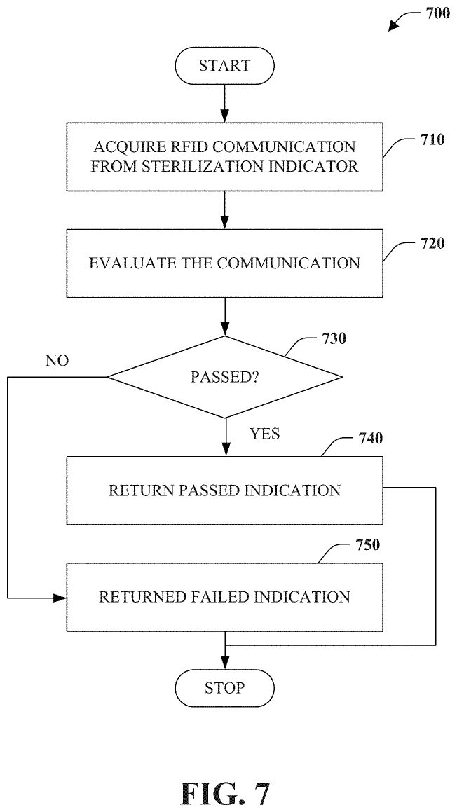

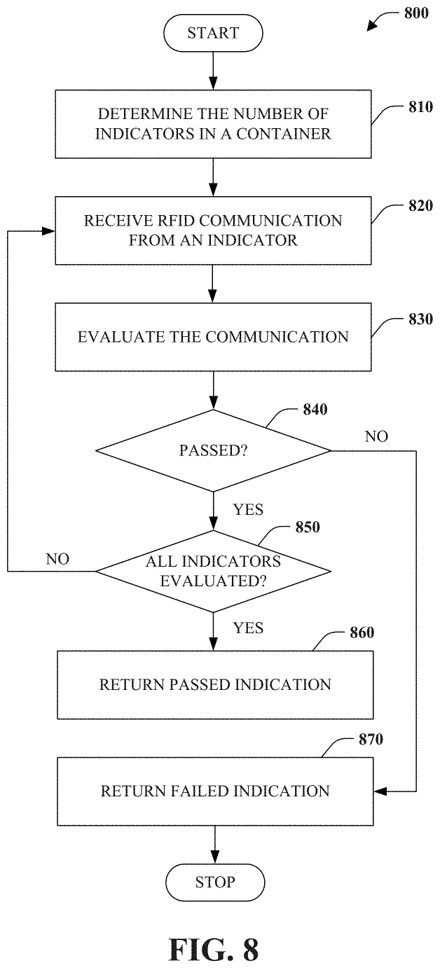

In view of the example systems described above methods that may be implemented in accordance with the disclosed subject matter will be better appreciated with reference to flow chart diagrams of FIGS. 7 and 8. While for purposes of simplicity of explanation, the methods are shown and described as a series of blocks. It is to be understood and appreciated that the disclosed subject matter is not limited by the order of the blocks, as some blocks may occur in different orders or concurrently with other blocks from what is depicted and described herein. Moreover, not all illustrated blocks may be required to implement the methods described hereinafter. Further, each block or combination of blocks can be implemented by computer program instructions that can be provided to a processor to produce a machine, such that the instructions executing on the processor create a means for implementing functions specified by a flow chart block.

FIG. 7 illustrates a method 700 of verifying a sterilization process in accordance with one embodiment. The method can be executed by a radio frequency identification (RFID) reader, for instance, comprising the reader component 130 and evaluator component 140 of FIG. 1.

At numeral 710, RFID communication is received, retrieved, or otherwise obtained or acquired from a sterilization process indicator. The process indicator can be in a container or the like with medical equipment subject to a sterilization process. An active sterilization process indicator can broadcast a signal utilizing an internal power source that can be detected and acquired. A passive sterilization process indicator does not have a power source but rather acquires energy to transmit a signal from a reader within range. Thus, the reader can request and receive a signal from the process indicator.

At numeral 720, the received communication can be evaluated. Evaluation can correspond to comparing input such as resistance or capacitance to one or more threshold values. For example, if a resistance process indicator is employed, communication can correspond to a value capturing resistance between two points. A high resistance relative to a predetermined value can indicate failure, while low resistance with respect to a predetermined value can denote success or a pass. For a capacitive process indicator, the capacitance can be received as the communication and compared with a predetermined threshold value to determine whether a sterilization process was successful or not.

At numeral 730, a determination is made as to whether or not the sterilization passed or not given observations captured by the process indicator and comparison with predetermined thresholds. If sterilization passes (“YES”), the method 700 returns a passed indication at 740 and terminates. If sterilization did not pass (“NO”), the method 700 returns a failure indication at 750 and terminates.

FIG. 8 depicts a method of sterilization verification 800. In one embodiment, the method 800 can be implemented and performed by an RFID reader.

At reference numeral 810, the number of indicators in a container is determined. Active process indicators broadcast their signal or communication. Accordingly, the number of indicators can be determined by counting the number of different detected broadcast signals. Actions can be similar for passive process indicators, except a reader can provide the power to communicate. Distinct communications can be counted to determine the number of indicators. It should also be appreciated that in addition to detecting the number of indicators in a container, the reader can identify the location of the indicators, for example, based on signal strength and the location of the reader.

At numeral 820, RFID communication is received, retrieved, or otherwise obtained or acquired from a process indicator. The communication can include process indicator unique identifier and value captured process indicator. For example, the value can correspond to the determined resistance or capacitance associated with the process indicator. In one embodiment, receiving the communication can be incorporated into determining the number of indicators. For example, communications can be received, and the number of communications received can be counted. Further, unique identifiers can be used to distinguish process indicators when counting.

The communication can be evaluated against one or more predetermined thresholds at 830. The predetermined thresholds can be set that indicate success or failure of a sterilization process. For example, a threshold resistance value can be set that indicates success or failure. Similarly, a capacitance threshold can be set that indicates whether the sterilization process passed or failed with respect to one process indicator.

At numeral 840, the method 800 determines whether sterilization passed based on evidence observed and captured by an indicator versus what is expected or required. If sterilization passed (“YES”), the method 800 continues at 850. If sterilization has not passed (“NO”), the method 800 can return a failure indication at 870 and subsequently terminate.

At 840, the method 800 determines whether all indicators have been evaluated. The determination can be made by comparing a record of the number of indicators evaluated or otherwise processed against the number of indicators determined. If all indicators have not been evaluated (“NO”), the method continues at 820, where RFID communication is acquired from a different indicator. If, at 850, it is determined that all indicators have been evaluated (“YES”), the method can proceed to 860, where a passed indication is returned, and the method terminates. In this manner, all indicators are evaluated, and if one does not pass, the overall evaluation does not pass. Of course, this determination can vary in other implementations. For example, if one process indicator does not pass, the overall process may still be deemed to have passed.

At numeral 870, the method 800 can include more information than a failure indication. In one instance, the unique identifier of the process indicator can be returned alone or in combination with the position of the process indicator in a container or tray.

Aspects of the subject disclosure pertain to the technical problem of determining conditions objects experienced in a sterilization container subject to a lethal process to remove, kill, or deactivate life forms, particularly microorganisms, and other biological agents before use and without exposing the objects to airborne contamination. The technical solution can include producing a process indicator that changes an electrical property of the process indicator in response to sterilization chamber conditions and communicating that electrical property by way of a radio frequency antenna outside the container to be evaluated to determine a state observed by the process indicator. The technical solution further concerns employing an amplifier, specialized antenna, or both to overcome signal interference from the container itself.

As used herein, the terms “component” and “system,” as well as various forms thereof (e.g., components, systems, sub-systems), are intended to refer to a computer-related entity, either hardware, a combination of hardware and software, software, or software in execution. For example, a component may be, but is not limited to being, a process running on a processor, a processor, an object, an instance, an executable, a thread of execution, a program, or a computer. By way of illustration, both an application running on a computer and the computer can be a component. One or more components may reside within a process or thread of execution, and a component may be localized on one computer or distributed between two or more computers.

As used herein, the term “infer” or “inference” generally refer to the process of reasoning about or inferring states of a system, a component, an environment, or a user from one or more observations captured by way of events or data, among other things. Inference may be employed to identify a context or an action or may be employed to generate a probability distribution over states, for example. An inference may be probabilistic. For example, a probability distribution over states of interest can be computed based on a consideration of data or events. Inference may also refer to techniques employed for composing higher-level events from a set of events or data. Such inference may result in the construction of new events or new actions from a set of observed events or stored event data, whether or not the events are correlated in close temporal proximity, and whether the events and data come from one or several events and data sources.

The conjunction “or” as used in this description and appended claims is intended to mean an inclusive “or” rather than an exclusive “or,” unless otherwise specified or clear from context. In other words, “‘X’ or ‘Y’” is intended to mean any inclusive permutations of “X” and “Y.” For example, if “‘A’ employs ‘X,’” “‘A employs ‘Y,’” or “‘A’ employs both ‘X’ and ‘Y,’” then “‘A’ employs ‘X’ or ‘Y’” is satisfied under any of the foregoing instances.

Furthermore, to the extent that the terms “includes,” “contains,” “has,” “having” or variations in form thereof are used in either the detailed description or the claims, such terms are intended to be inclusive in a manner similar to the term “comprising” as “comprising” is interpreted when employed as a transitional word in a claim.

To provide a context for the disclosed subject matter, FIG. 8 and the following discussion are intended to provide a brief, general description of a suitable environment in which various aspects of the disclosed subject matter can be implemented. The suitable environment, however, is solely an example and is not intended to suggest any limitation as to the scope of use or functionality.

While the above-disclosed system and methods can be described in the general context of computer-executable instructions of a program that runs on one or more computers, those skilled in the art will recognize that aspects can also be implemented in combination with other program modules or the like. Generally, program modules can include routines, programs, components, and data structures, among other things, that perform particular tasks or implement particular abstract data types. Moreover, those skilled in the art will appreciate that the above systems and methods can be practiced with various computer system configurations, including single-processor, multi-processor or multi-core processor computer systems, mini-computing devices, server computers, as well as personal computers, hand-held computing devices (e.g., personal digital assistant (PDA), smartphone, tablet, watch . . . ), microprocessor-based or programmable consumer or industrial electronics, and the like. Aspects can also be practiced in distributed computing environments where tasks are performed by remote processing devices linked through a communications network. However, some, if not all aspects, of the disclosed subject matter can be practiced on stand-alone computers. Program modules may be located in one or both of local and remote memory devices in a distributed computing environment.

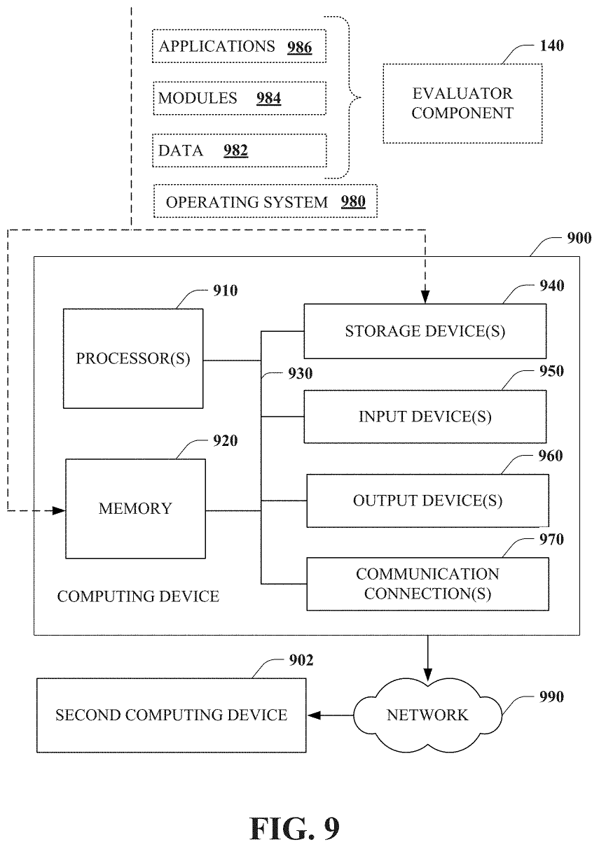

With reference to FIG. 9, illustrated is an example computing device 900 (e.g., desktop, laptop, tablet, watch, server, hand-held, programmable consumer or industrial electronics, set-top box, game system, compute node). The computing device 900 includes one or more processor(s) 910, memory 920, system bus 930, storage device(s) 940, input device(s) 950, output device(s) 960, and communications connection(s) 970. The system bus 930 communicatively couples at least the above system constituents. However, the computing device 900, in its simplest form, can include one or more processors 910 coupled to memory 920, wherein the one or more processors 910 execute various computer-executable actions, instructions, and or components stored in the memory 920.

The processor(s) 910 can be implemented with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any processor, controller, microcontroller, or state machine. The processor(s) 910 may also be implemented as a combination of computing devices, such as a combination of a DSP and a microprocessor, a plurality of microprocessors, multi-core processors, or one or more microprocessors in conjunction with a DSP core, or any other such configuration. In one embodiment, the processor(s) 910 can be a graphics processor unit (GPU) that performs calculations for digital image processing and computer graphics.

The computing device 900 can include or otherwise interact with a variety of computer-readable media to facilitate control of the computing device to implement one or more aspects of the disclosed subject matter. The computer-readable media can be any available media that is accessible to the computing device 900 and includes volatile and nonvolatile media, and removable and non-removable media. Computer-readable media can comprise two distinct and mutually exclusive types, namely storage media and communication media.

Storage media includes volatile and nonvolatile, removable and non-removable media implemented in any method or technology for storage of information such as computer-readable instructions, data structures, program modules, or other data. Storage media includes storage devices such as memory devices (e.g., random access memory (RAM), read-only memory (ROM), electrically erasable programmable read-only memory (EEPROM) . . . ), magnetic storage devices (e.g., hard disk, floppy disk, cassettes, tape . . . ), optical disks (e.g., compact disk (CD), digital versatile disk (DVD) . . . ), and solid state devices (e.g., solid state drive (SSD), flash memory drive (e.g., card, stick, key drive . . . ) . . . ), or any other like mediums that store, as opposed to transmit or communicate, the desired information accessible by the computing device 900. Accordingly, storage media excludes modulated data signals as well as that described with respect to communication media.

Communication media embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared, and other wireless media.

The memory 920 and storage device(s) 940 are examples of computer-readable storage media. Depending on the configuration and type of computing device, the memory 920 may be volatile (e.g., random access memory (RAM)), non-volatile (e.g., read only memory (ROM), flash memory . . . ), or some combination of the two. By way of example, the basic input/output system (BIOS), including basic routines to transfer information between elements within the computing device 900, such as during start-up, can be stored in nonvolatile memory. In contrast, volatile memory can act as external cache memory to facilitate processing by the processor(s) 910, among other things.

The storage device(s) 940 include removable/non-removable, volatile/non-volatile storage media for storage of vast amounts of data relative to the memory 920. For example, storage device(s) 940 include, but are not limited to, one or more devices such as a magnetic or optical disk drive, floppy disk drive, flash memory, solid-state drive, or memory stick.

Memory 920 and storage device(s) 940 can include, or have stored therein, operating system 980, one or more applications 986, one or more program modules 984, and data 982. The operating system 980 acts to control and allocate resources of the computing device 900. Applications 986 include one or both of system and application software and can exploit management of resources by the operating system 980 through program modules 984 and data 982 stored in the memory 920 or storage device(s) 940 to perform one or more actions. Accordingly, applications 986 can turn a general-purpose computer 900 into a specialized machine in accordance with the logic provided thereby.

All or portions of the disclosed subject matter can be implemented using standard programming or engineering techniques to produce software, firmware, hardware, or any combination thereof to control the computing device 900 to realize the disclosed functionality. By way of example and not limitation, all or portions of the evaluator 140 can be, or form part of, the application 986, and include one or more modules 984 and data 982 stored in memory or storage device(s) 940 whose functionality can be realized when executed by one or more processor(s) 910.

In accordance with one particular embodiment, the processor(s) 910 can correspond to a system on a chip (SOC) or like architecture including, or in other words integrating, both hardware and software on a single integrated circuit substrate. Here, the processor(s) 910 can include one or more processors as well as memory at least similar to the processor(s) 910 and memory 920, among other things. Conventional processors include a minimal amount of hardware and software and rely extensively on external hardware and software. By contrast, an SOC implementation of a processor is more powerful, as it embeds hardware and software therein that enable particular functionality with minimal or no reliance on external hardware and software. For example, the evaluator 140 or functionality associated therewith can be embedded within hardware in an SOC architecture.

The input device(s) 950 and output device(s) 960 can be communicatively coupled to the computing device 900. By way of example, the input device(s) 950 can include a pointing device (e.g., mouse, trackball, stylus, pen, touchpad), keyboard, joystick, microphone, voice user interface system, camera, motion sensor, and a global positioning satellite (GPS) receiver and transmitter, among other things. The output device(s) 960, by way of example, can correspond to a display device (e.g., liquid crystal display (LCD), light emitting diode (LED), plasma, organic light-emitting diode display (OLED) . . . ), speakers, voice user interface system, printer, and vibration motor, among other things. The input device(s) 950 and output device(s) 960 can be connected to the computing device 900 by way of wired connection (e.g., bus), wireless connection (e.g., Wi-Fi, Bluetooth), or a combination thereof.

The computing device 900 can also include communication connection(s) 970 to enable communication with at least a second computing device 902 by means of a network 990. The communication connection(s) 970 can include wired or wireless communication mechanisms to support network communication. The network 990 can correspond to a local area network (LAN) or a wide area network (WAN) such as the Internet. The second computing device 902 can be another processor-based device with which the computing device 900 can interact. For example, the computing device 900 can correspond to an RFID reader that can be communicatively coupled with a second computing device 902. In one instance, functionality of the RFID reader can be split between the computing device 900 and the second computing device 902. Alternatively, the computing device 900 can implement the RFID reader functionality and save the data to a database executing on the second computing device 902.

In accordance with one embodiment, an indicator tag or the like can be implemented as a system on a chip that can observe conditions and make determinations itself as to whether or not sterilization passed or failed. In response to interrogation, a simple pass or fail can be returned alone or in conjunction with other information.

What has been described above includes examples of aspects of the claimed subject matter. It is, of course, not possible to describe every conceivable combination of components or methodologies for purposes of describing the claimed subject matter, but one of ordinary skill in the art may recognize that many further combinations and permutations of the disclosed subject matter are possible. Accordingly, the disclosed subject matter is intended to embrace all such alterations, modifications, and variations that fall within the spirit and scope of the appended claims.

What is claimed is:

1. A process indicator system, comprising
a dye material that has a melting point at a predetermined sterilization temperature and can be forced along a defined path by steam that alters an electronic property value of a process indicator;
a radio frequency antenna that communicates the electronic property value of the process indicator wirelessly; and
an amplifier coupled with the radio frequency antenna that amplifies the radio frequency.

2. The system of claim 1, further comprising a flexible metal wire of a predetermined length that couples the radio frequency antenna to the amplifier at a distance.

3. The system of claim 1, wherein the dye material conducts electricity.

4. The system of claim 3, wherein the electronic property is resistance, and the resistance between two metal portions varies based on a position of the dye material along the defined path.

5. The system of claim 4, further comprising:
a first dielectric polymer layer, wherein the two metal portions and the dye material are overlaid on the first dielectric polymer layer; and
a second dielectric polymer layer overlaid on the two metal portions and the dye material.

6. The system of claim 1, wherein the dye material has a capacitive property.

7. The system of claim 6, further comprising:
a first metal layer;
a first dielectric polymer layer overlaid on the first metal layer;
the dye material overlaid on the first dielectric polymer layer;
a second dielectric polymer layer overlaid on the dye material; and
a second metal layer overlaid on the second dielectric polymer layer.

8. The system of claim 7, wherein the electronic property is capacitance and the capacitance between the first metal layer and the second metal layer varies based on a position of the dye material along the defined path.

9. The system of claim 1, further comprising a printed indication of a location of the dye material on the path that indicates that a sterilization condition has been met.

10. The system of claim 1, further comprising a power source that powers broadcast of the electronic property value.

11. A method, comprising:
executing, on a processor, instructions that cause the processor to perform operations associated with process indicator evaluation, the operations comprising:
reading an amplified radio frequency of an electronic property value of a process indicator from an antenna coupled to the process indicator inside a sterilization container, wherein the process indicator includes an ink material with a melting point at a predetermined sterilization temperature that moves along a predefined path and alters the electronic property value;
evaluating the electronic property value with respect to one or more predetermined thresholds; and
conveying, for display on a display device, either pass or fail based on a result of the evaluating.

12. The method of claim 11, wherein evaluating the electronic property value comprises comparing resistance as the electronic property value to a predetermined resistance threshold that indicates conditions for sterilization were satisfied.

13. The method of claim 11, wherein evaluating the electronic property value comprises comparing capacitance as the electronic property value to a predetermined capacitance threshold indicating conditions for sterilization were satisfied.

14. The method of claim 11, the operations further comprising reading the electronic property value from a radio frequency antenna outside the sterilization container connected by a flexible wire to the process indicator inside the sterilization container.

15. The method of claim 11, the operations further comprising determining a number of process indicators inside the sterilization container through wireless communication.

16. The method of claim 15, the operations further comprising:
evaluating the electronic property value of the number of process indicators inside the sterilization container; and
conveying fail as the result when any one of the number of process indicators fails.

17. The method of claim 11, the operations further comprising:
determining a location of the process indicator within the sterilization container based on reading position and signal strength; and
conveying, for display on the display device, the location of the process indicator.

18. A process indicator apparatus, comprising
a dye material that has a melting point at a predetermined sterilization temperature and can be forced along a defined path by steam that alters an electronic property value of a process indicator;
a radio frequency antenna that communicates the electronic property value of the process indicator wirelessly; and
an amplifier that amplifies the radio frequency, wherein the amplifier is coupled to the radio frequency antenna with a predetermined length of flexible wire that allows the radio frequency antenna to be positioned outside a sterilization container while the dye material and amplifier are inside the sterilization container.

19. The process indicator apparatus of claim 18, wherein the electronic property is resistance, and the resistance between two metal portions varies based on a position of the dye material along the defined path.

20. The process indicator apparatus of claim 18, wherein the electronic property is capacitance and the capacitance between a first metal layer and a second metal layer varies based on a position of the dye material along the defined path.
